(19) United States
(12) Patent Application Publication
(10) Pub. No.: US 2006/0088599 A1
(43) Pub. Date: Apr. 27, 2006

(54) Amino functionalized ORMOSIL nanoparticles as delivery vehicles

(76) Inventors: Paras N. Prasad, Williamsville, NY (US); Earl J. Bergey, South Dayton, NY (US); Purnendu Dutta, Williamsville, NY (US); Dhruba J. Bharali, Amherst, NY (US); Michal Stachowiak, East Amherst, NY (US); Tymish Ohulchanskyy, Kenmore, NY (US); Ilona Klejbor, Gdynia (PL); Indrajit Roy, Amherst, NY (US)

Correspondence Address: HODGSON RUSS LLP, ONE M & T PLAZA, SUITE 2000, BUFFALO, NY, 14203-2391, US

(21) Appl. No.: 11/195,066

(22) Filed: Aug. 2, 2005

Related U.S. Application Data
(60) Provisional application No. 60/598,092, filed on Aug. 2, 2004.

(51) Int. Cl. A61K 48/00; A61K 9/50; A61K 9/16; C12N 15/87

(52) U.S. Cl. 424/490; 514/44; 435/459; 977/916

(57) ABSTRACT

Provided are amino functionalized ORMOSIL nanoparticles. Also provided are compositions comprising such particles and compositions in which the nanoparticles are complexed to polynucleotides. The complexing of polynucleotides to the amino functionalized ORMOSIL nanoparticles protects the polynucleotides from environmental damage. These complexes can be used for delivery of polynucleotides to cells.

This application claims priority to provisional application Ser. No. 60/598,092, filed on Aug. 2, 2004, the disclosure of which is incorporated herein by reference.

### FIELD OF THE INVENTION

The present invention relates generally to the field of non-viral based gene therapy.

### BACKGROUND OF THE INVENTION

Treatment of genetic disorders has advanced tremendously with the ability to identify the specific genes whose defect or absence is responsible for the particular pathological condition. Such therapeutic genes, if could be carefully delivered inside the cells with deficient or defective genes, will provide a very useful method of treatment. In order for genes to enter cells, they require a vector which can securely bind with the therapeutic genes or encapsulate the genes and transgress the cell membrane. Ultimately the genes are released inside the cell and allowed to multiply, causing transfection. Genes, by themselves, are unable to enter the cells.

Viral vectors have been used in many experiments for successful transfection. Viruses can easily enter cells carrying the genetic payload. However, it is extremely difficult to produce a stable strain of harmless virus for this purpose and have it commercially available. Furthermore, the viruses are very unpredictable in their behavior after administration, due to possible mutagenesis and transformation into virulent forms. Deaths have occurred in human trials leading to a halt in further use of viral vectors for gene transfection.

Many diseases are currently being investigated in transgenic animals by either knocking out (KO) or knocking down (KD) specific genes, or by expressing mutant genes. Development of such transgenic animals requires specially equipped animal facilities, may take few years and is expensive. Hence, the transgenic animal approach is not practical for screening large numbers of pathogenic and therapeutic genes. Also, it is often unclear whether the observed biological changes or pathologies reflect transgene action in mature nervous tissue or its impact on animal development.

Extensive research has been in progress for the quest of non-viral delivery vehicle. Such non-viral delivery vehicles include ceramic nanoparticles, polyethyleneimine, and polymeric anoparticles. These delivery vehicles are difficult to produce, have difficulty in the release of DNA and poor transfection efficiency, and have exhibited in vivo toxicity.

### SUMMARY OF THE INVENTION

The present invention provides amino functionalized organically modified silica (amino functionalized ORMOSIL) nanoparticles and complexes of such particles with polynucleotides.

These poynucleotide-amino functionalized ORMOSIL complexes can be used for delivery of the polynucleotides to cells. For example, these complexes can be used as a non-viral gene transfection vehicle. The amino functionalized ORMOSIL particles provide researchers and clinicians with an in vivo mechanism to insert genes into host tissue at efficiencies comparable or better than current technology, without the side effects associated with these viral and chemical methodologies.

In one embodiment, this invention provides the synthesis of cationic ORMOSIL nanoparticles and complexing of DNA to aminofunctionalized ORMOSIL with such complexes being capable of protecting the polynucleotide from environment damage.

In another embodiment, DNA complexed to the amino functionalized ORMOSIL particles was shown to be protected from environmental damage and used for in vivo transfection of brain neurons and progenitor cells. The DNA-amino functionalized ORMOSIL nanoparticle complex is demonstrated herein to have in vivo transfection efficiencies equal to or greater than that of the current in vivo technology (polyethyleneimine and viral-based mechanisms).

### BRIEF DESCRIPTION OF THE DRAWINGS

FIG. 1—Scheme depicting the synthesis dye doped ORMOSIL nanoparticles.

FIG. 2—Release kinetics of amphiphilic dye (Rh6G; curves 1) and hydrophobic dye (HPPH; curves 2) from ORMN20 nanoparticles.

FIG. 3—Image of agarose gel electrophoresis of plasmid DNA, free as well as complexed with ORMOSIL nanoparticles. Lane 1: λ-DNA Hind III digest, Lane 2: peGFP, Lane 3: ORMN20+peGFP, Lane 4: ORMA20+peGFP, Lane 5: ORMA40+peGFP, Lane 6: peGFP+DNase1, Lane 7: ORMN20+peGFP+DNase1, Lane 8: ORMA20+peGFP+DNase1 Lane 9: ORMA40+peGFP+DNase1, Lane 9: ORMA40+peGFP+DNase1.

FIG. 4—COS-1 cells transfected with eGFP vector delivered with amino functionalized ORMOSIL nanoparticles. Transmission microscopic image (blue) and fluorescence (green) image is shown as a combined image.

FIG. 5 ORMOSIL nanoparticle transfection in the SNc. (A) DNA-free af-ORMOSIL injection showing no substantial immunostaining for EGFP. (B-E) Injection of af-ORMOSIL-pEGFP-N2 complex into SNc. (B) Multiple cells with typical dopaminergic neuron morphology are immunostained positive for EGFP. (C) No immunostaining is observed without primary anti-EGFP Ab. (D) EGFP immunostaining of neuron-shaped cells (higher magnification). (E) Transfected EGFP (green) is expressed in TH-immunopositive (red) dopaminergic neuron.

FIG. 6—Expression of EGFP in multiple brain areas after injection of ORMOSILpEGFP-N2 into the brain LV. (A and B) Control af-ORMOSIL nanoparticles. (A) The region surrounding the LV. Str, striatum; Sep, septum; cc, corpus callosum. (B) The hippocampal region adjacent to the ventricle. (C—F) af-ORMOSIL-pEGFP-N2 particles. Injection resulted in EGFP immunostaining of the neuron-shaped cells in dorsal lateral (d), lateral (1), and medial (m) septal nuclei (C); in the adjacent striatal region of the brain (D); cingulate and motor cortex (E); and pyramidal neurons of the CA3 hippocampal region (F).

FIG. 7-Transfection of ORMOSIL-pEGFP-N2 complex into the LV cells of the SVZ in mice were transfected with ORMOSIL-pEGFP-N2 by injection into the brain LV. (A and B) Seven days postmortem EGFP immunostaining is shown at low magnification (A) and at higher magnification (B) of the positive region to visualize transfected cells. (C and D) In vivo imaging of EGFP fluorescence in cells in the LV. Ten days after transfection, mice were subjected to the second stereotaxic surgery, and a miniature fiber-optic Cell-viZio probe was inserted into the anterior dorsal region (C) or the posterior region (D) of the LV>15 μm from the medial ventricular wall. Dynamic sequences were recorded, and selected frames are shown.

FIG. 8—Modulation of cell proliferation by using ORMOSIL transfection of nonmembrane_nucleus-targeted FGFR1(SP-/NLS). Control af-ORMOSIL (A, C, and E) or af-ORMOSIL/pFGFR1 (SP-INLS) (B, D, and F) was injected into the anterior region of the brain LV. Seven days later, the animals were injected with BrdUrd (i.p.) and were perfused 5 h later. Sagittal brain sections were immunostained for FGFR1 or DNA that had incorporated BrdUrd. (A and B) Immunostaining of SVZ with FGFR1 McAb6. (C and D) BrdUrd immunostaining of cell nuclei in SVZ and adjacent tissue. (E and F) BrdUrd immunostaining of cells in the rostral migratory stream close to SVZ.

### DETAILED DESCRIPTION OF THE INVENTION

Gene delivery is an area of considerable current interest, where genetic materials (DNA, RNA, oligonucleotides) have been used as molecular medicine and are delivered to specific cell types in order to either inhibit some undesirable gene-expression or to synthesize therapeutic proteins. Owing to the risk factors (pathogenicity, immunogenicity etc.) associated with viruses as gene-carriers (viral vectors), a major emphasis has been given towards the development of synthetic nanoparticles bearing cationic groups as non-viral vectors. In the present invention, we have produced ultra-fine silica nanoparticles with surfaces functionalized by cationic-amino groups and shown to not only bind and protect plasmid DNA from enzymatic digestion, but also to transfect cultured cells, in vitro and neurons, in vivo. To our knowledge, there is no previous report regarding successful use of such nanoparticles as gene-carriers in vivo.

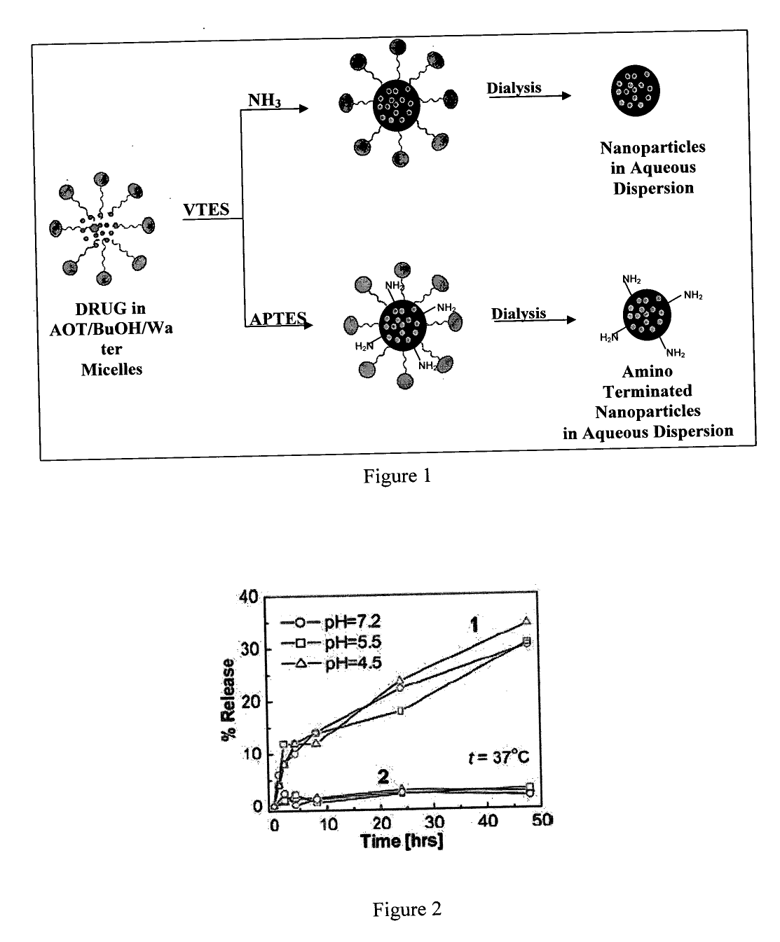

Organically modified silica (ORMOSIL) nanoparticles have the potential to overcome the many limitations of their ‘un-modified’ silica counterparts. ORMOSIL nanoparticles have the potential to overcome the many limitations of their ‘un-modified’ silica counterparts. Organically modified silica nanoparticles are synthesized from the silica precursors where one or two (out of four) of the alkoxysilane groups has been replaced by organic groups like vinyl, phenyl, octyl etc. Subsequently, upon condensation of the precursors, the organic group/s gets incorporated within the network of the synthesized nanoparticles (FIG. 1).

The presence of both hydrophobic and hydrophilic groups on the precursor alkoxy-organosilane helps them to self-assemble as both normal micelles and reverse micelles under appropriate conditions. The resulting micellular cores can be loaded with DNA (or other nucleic acids). The polynucleotides is/are held on the outside of nanoparticles. While not intending to be bound by any particular theory, it is believed that the nucleic acid molecules are held on the outside of the particles by electorstatic interaction resulting in amino functionalized ORMOSIL-nucleic acid nanocomplexs.

The ORMOSIL particles of the present invention have a number of advantages, (a) they can be loaded with either hydrophilic or hydrophobic molecules, (b) they can be precipitated in oil-in-water microemulsions, where corrosive solvents like cyclohexane and complex purification steps like solvent evaporation, ultra-centrifugation etc., can be avoided, (c) their organic groups can be further modified for the attachment of targeting molecules, and (d) they are possibly bio-degraded through the biochemical decomposition of the Si—C bond. The presence of the organic group also reduces the overall rigidity and density of the particle, which is expected to enhance the stability of such particles in aqueous systems against precipitation.

Nucleic acids that can be delivered using this method include both single and double stranded nucleic acids and can be DNA, RNA and DNA-RNA hybrids. The nucleic acids can be oligonucleotides or larger nucleic acids, such as plasmids or cosmids, or artificial chromosomes, such as yeast artificial chromosomes (“YACs”) or bacterial artificial chromosomes (“BACs”). Exemplary plasmids include of pEGFP, pBK-Q20-HA; pBK-Q127-HA and pcDNA3.1-FGFR1(SP-/NLS)

For the delivery of DNA to cells, the advantages of the present invention include: 1) amino functionalized ORMOSIL nanoparticles protect DNA from environmental degradation during in vivo transfection processes to produce efficient transfection that is at least as efficient as currently used methods. 2) DNA-amino functionalized ORMOSIL nanoparticles have the potential to be biocompatible in host system for efficient in vivo transfection of targeted tissues. 3) DNA-amino functionalized ORMOSIL nanoparticles can be tailor-made to target specific cells using chemical and biological ligands. 4) DNA-amino functionalized ORMOSIL particles can be utilized in the identification of genes and genetic mechanisms involved in the pathogenesis of diseases of the neurological system. 5) amino functionalized ORMOSIL nanoparticles can act as a vehicle for RNA-mediated interference (RNAi). 6) Amino functionalized ORMOSIL nanoparticles can provide a transfection mechanism for gene therapies for brain cancers as well as other diseases of the nervous system. 7) amino functionalized ORMOSIL nanoparticles could provide a novel mechanism for systemic use in in vivo transfection 8) DNA-amino functionalized ORMOSIL nanoparticles can facilitate development of new disease models in animal systems.

ORMOSIL is well known in the art. The present invention provides ORMOSIL nanoparticles (10-100 nm) which are relatively easy to produce on a mass scale. In the present invention, the surfaces of these particles are modified further to make it positively charged by amino functionalizing it during synthesis. This process enhances binding with negatively charged nucleic acids for successful carriage inside the cells. This process of loading the nanoparticles with a nucleic acid, such as DNA, for transfection is considerably simpler than the production of other transfectable material and its encapsulation in viral particles. This binding provides protection of the sensitive DNA structure to environment insult during the process involved in in vivo transfer. This DNA-nanoparticle complex is stable and easily stored until use in transfection or transformation. As used herein, the terms “transformation” and “transfection” are intended to refer to a variety of art-recognized techniques for delivering a nucleic acid (e.g., RNA or DNA) into a cell, including calcium phosphate or calcium chloride co-precipitation, DEAE-dextran-mediated transfection, lipofection, or electroporation. Suitable methods for transforming or transfecting cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

In one embodiment, an amino functionalized ORMOSIL-polynucleotide complex can be used to transform mammalian cells. In order to identify and/or select cells comprising the delivered polynucleotide, a gene that encodes a selectable marker (e.g., resistance to antibiotics) can be introduced into the host cells along with the gene of interest. Various selectable markers include those that confer resistance to drugs, such as G418, hygromycin and methotrexate. Additionally, detectable markers can be added to identify cells that comprise the delivered polynucleotide. Suitable detectable markers include those that can be visually detected, such as β-galactosidase or green fluorescent protein.

In another embodiment, the invention pertains to cells into which a nucleic acid-amino functionalized ORMOSIL complex has been delivered.

Comparison of the literature liposome data with our results of transfecting mouse brain tissue with the amino functionalized ORMOSIL nanoparticles clearly indicate that DNA-amino functionalized ORMOSIL complexes allow transfections that are more effective. The amino functionalized ORMOSIL nanoparticle-mediated transfection of glioma tumors in combination with plasmids expressing therapeutic fusion proteins that are released from transfected cells would allow more effective anti-glioma therapies than are currently available.

These DNA loaded amino functionalized ORMOSIL nanoparticles are taken up the cells. Once inside the cells, the nanoparticle-gene complex breaks down, releasing the genes, which are subsequently transported to the nucleus for transcription. We have utilized polyethyleneimine, calcium phosphate nanoparticle and HSV as vectors for in vivo gene transfer in brain cells by direct injection in different areas of brain with concentration of neuronal cells, e.g., substantia nigra and striatum. Transfection was demonstrated in each case, but was suboptimal, transfecting significantly lower number of cell and expressing lower levels of the transgene. In addition, these procedures have significant side effects, which can compromise to target host. These include: (1) Carrier toxicity, (2) Injury due to immunological side effects, and (3) Conversion to pathogenic form during transfection process.

An effective gene transfer into the central nervous system (CNS) using amino functionalized ORMOSIL nanoparticles (see the above), combined with the most recent molecular technologies (i.e. small inhibitory RNA) would allow developing and testing the KD or DMN genetic disease models. Being able to transfect genes into specific CNS regions and at specific times will allow one to produce produce biological/pathological effects in selected CNS regions and cells and at an optimal time. DNA transfections can be done for CNS of rodents and other animals including primates and humans in which the disease processes often have different clinical manifestations and respond differently to therapeutic agents (are more akin to the human diseases). For instance, the amino functionalized ORMOSIL nanoparticles could be used to model the Huntington disease by transfecting a mutated Huntington gene into brain basal ganglia. Amyotropic lateral sclerosis (ALS) could be modeled by transfecting superoxide dismutase 1 gene with different mutations. Diseases that appear to have a diverse genetic background, such as Parkinson Disease (PD), could be modeled by transfecting specific adult brain regions with mutant alpha-synuclein gene, by blocking specific growth factor signaling (i.e., FGF, GDNF) using DMN growth factor receptor mutant genes, or by KD of the parkin gene using small interfering-RNA technology as described below.

Amino functionalized ORMOSIL nanoparticles can also act as a vehicle for RNA-mediated interference (RNAi). RNAi has recently emerged as a powerful tool for regulating mammalian gene expression. Duplexes of 21-nucleotide-long double-stranded small inhibitory RNA (siRNAs) effectively suppress gene expression by preventing translation or inducing degradation of the specific RNAs targeted by siRNA. Viral-based vector systems for the long-term delivery of RNA hairpins have been developed, yet they require expertise in viral production and transduction. In addition, the pathological side effects of viral vectors in the NS may prevent their use in some experiments and in human therapies. The new ORMOSIL particle nanotechnology would allow relatively simple plasmid-based system for delivering DNA for small inhibitory RNA hairpins for the generation of gene knockdown. Using amino functionalized ORMOSIL nanoparticles in conjunction with plasmids expressing hairpin-shaped double-stranded siRNA, it should possible to turn off specific individual as well as groups of genes in order to analyze their role in development. To translate the RNAi technology to medical use, an immediate challenge is to determine the effectiveness of siRNAs in living animals. As a step towards this goal, the amino functionalized ORMOSIL nanoparticles offer an effective new technology for transfecting plasmids expressing siRNAs into the CNS or CNS neoplasms.

Gene therapies for CNS injuries and stroke would have a significant impact on the health profession and individuals suffering from injuries or stroke and have exceptionally high social costs. Several experimental strategies have been proposed to minimize tissue damage and to enhance axonal growth and regeneration after spinal cord or brain injury. The introduction of genes using amino functionalized ORMOSIL nanoparticles that can stimulate axonal growth and neurogenesis to augment morphological and functional recovery is one such strategy. Immediately after spinal cord or brain injury, the initial mechanical damage is followed by a cascade of harmful secondary events that include the formation of free radicals, detrimental inflammatory responses, and death of neurons and glia. At this time point, gene transfer interventions could address those processes to preserve axons and neurons and maximize their function, while limiting further neuronal and glial loss. The stability and ease of loading polynucleotide complexed amino functionalized ORMOSIL nanoparticles could effective provide this rapid transfection to limit neuronal and glial loss. At later time points, amino functionalized ORMOSIL based interventions could be developed to stimulate neurogenesis, axonal growth, neutralize potential growth inhibitory molecules, to guide axons to their targets and to establish new functional synapses. Hence, the safety profile of gene therapy would likely be higher with the nonviral than with viral technologies. The amino functionalized ORMOSIL-mediated gene transfer would to be well suited for these applications.

The present invention provides data from an established animal model for transfection of cells in the brain. Because significant bank of knowledge exists as to the efficacy of other transfixion technology, this allows for comparative studies without having to fully developing the methodology and characterization of the viral and chemical transfection systems. Our current studies have not indicated any significant in vivo adverse response to the use of amino functionalized ORMOSIL nanoparticles for transfixion of the cells in the brain. These include the length of expression of the gene (long lasting or transient). Transient transfection is sufficient to study the role of genes, in vivo, in development and in the treatment of brain/spinal cord injuries, stroke or cancer. The longer treatment of chronic neurodegenerative can benefit from the long lasting expression of the transgene. We have found that a gene transfected using the amino functionalized ORMOSIL-DNA nanocomplexes as described here is expressed for at least 21 days. In this experiment, amino functionalized ORMOSIL/Plasmid nanocomplexes were injected in the brain. At 21 days, mice were perfused and were found to be positive for the gene product. These animals did not show any overt toxic effects over this period of time. Our previous studies have shown that polyethyleneimine-mediated transfection shows long lasting expression of the transgene (at least 2-3 months). Having demonstrated that the amino functionalized ORMOSIL transfection system allows for a several-fold more efficient transfection than PEI, a similar long lasting transfection using amino functionalized ORMOSIL would be predicted and without the toxic side effects known to be associated with PEI.

### EXAMPLE 1

Synthesis and Characterization of Drug-Loaded Silica-Based Nanoparticles

The nanoparticles were synthesized in the non-polar core of AOT/DMSO/water micelles. Typically, the micelles were prepared by dissolving a fixed amount of AOT and 1-butanol in 20 mL of double distilled water by vigorous magnetic stirring. Then, 200 μl of neat triethoxyvinylsilane was added to the micellular system, and the resulting solution was stirred for ˜30 minutes. The ORMOSIL nanoparticles were then precipitated by adding aqueous ammonia solution or 3-aminopropyltriethoxysilane and stirring for about 20 hours. The entire reaction is carried out at room temperature.

During the synthesis, when ammonia is used to condense VTES, non-amino terminated nanoparticles form (e.g. 3ORM2N2) and when APTES is used to condense VTES, amino-terminated (amino functionalized) ORMOSIL nanoparticles form (e.g 3ORM2A2). The nanoparticles are termed herein as ORMOSIL, irrespective of whether they are amino-terminated or not.

At the end of the process, a blue-white translucency, indicating the formation of nanoparticles, was observed. After the formation of the nanoparticles, surfactant AOT and co-surfactant 1-butanol were removed by dialyzing the solution against water in a 12-14-kDa cutoff cellulose membrane for 50 hrs. The dialyzed solution was then filtered through a 0.2 μm cut-off membrane filter (Nalgene) and used directly for experimentation. The composition of different particle systems and the scheme of synthesis are given in Table 1 and FIG. 1, respectively. Transmission electron microscopy (TEM) was employed to determine the morphology and size of the aqueous dispersion of nanoparticles, using a JEOL JEM 2020 electron microscope, operating at an accelerating voltage of 200 kV. ORMOSIL particles were found to be monodispersed and uniform in size as determined by the synthetic protocol. Where fluorescent dye was incorporated for determination of DNA binding to ORMOSIL nanoparticles, fluorescence spectra were taken on a Fluorolog-3 spectrofluorometer (Jobin Yvon).

Without being bound by any particular theory, it is considered that the size and surface charge on the nanoparticles determine the transfection efficiency, the surface charge being the dominant one. The surface charge should be just positive enough to condense the negatively charged DNA, while too much excess positive charge might hinder the intracellular release of the polynucleotide. We have seen by X-ray photoelectron spectroscopy that with the increasing concentration of the amine-bearing ORMOSIL precursor (3-aminopropyltriethoxy silane, APTES) the amount of nitrogen atoms increases on the nanoparticles, which suggests increasing amino-functionality.

ORMOSIL precursors that are preferable are vinyltriethoxysilane (VTES) and phenyltrimethoxysilane (PTMS), and the amino-bearing precursor being 3-aminopropyltriethoxy silane (APTES). While longer carbon chain containing compounds (4-10 carbons) can also be used, APTES (3-carbon) has been found to be preferable as it holds the nucleic acid molecules close to the nanoparticle. Once the nanoparticles have been purified, they can be diluted or buffer-exchanged with buffers like PBS. The variations of these parameters and identification of optimal conditions are within the purview of those skilled in the art.

### EXAMPLE 2

Elemental Analysis (XPS)

For elemental analysis of solid samples, the nanoparticles in aqueous dispersion (after dialysis) were centrifuged and dried in an oven (1 hour at 80° C.). The dried samples were crushed to fine powder and were spread on a sample holder. A Physical Electronics Model (Perkin Elmer) 5300 X-Ray Photoelectron Spectrometer (XPS) was used for the elemental analysis of the sample. X-rays were generated with MG and Ti targets while ejected electrons were analyzed with a hemispherical analyzer. Data analysis is performed on a Pentium II 350 MHz computer connected to the instrument with a RBD manufactured interface control. The results of this analysis are shown in Table 2.

TABLE 1

| | AOT | nBuOH | Water | DMSO* | VTES | NH₃ | APTES | Diameter |
|---|---|---|---|---|---|---|---|---|
| Name | (g) | (μL) | (mL) | (μL) | (μL) | (μL) | (μL) | (nm) |
| 3ORM2N20 | 0.33 | 600 | 20 | 100 | 200 | 20 | — | 10-15 |
| 3ORM2A20 | 0.33 | 600 | 20 | 100 | 200 | — | 20 | 10-15 |
| 4ORM2N20 or | 0.44 | 800 | 20 | 100 | 200 | 20 | — | 15-20 |
| ORMN20 | | | | | | | | |
| 4ORM2A20 or | 0.44 | 800 | 20 | 100 | 200 | — | 20 | 15-20 |
| ORMA20 | | | | | | | | |
| 4ORM2A40 or | 0.44 | 800 | 20 | 100 | 200 | — | 40 | 15-20 |
| ORMA40 | | | | | | | | |
| 4ORM3N20 | 0.44 | 800 | 20 | 100 | 300 | 20 | — | 25-30 |
| 4ORM5N20 | 0.44 | 800 | 20 | 100 | 500 | 20 | — | 40-45 |
| 6ORM2N20 | 0.66 | 1200 | 20 | 100 | 200 | 20 | — | 40-45 |
| 6ORM3N20 | 0.66 | 1200 | 20 | 100 | 300 | 20 | — | 65-75 |
| 8ORM2N20 | 0.88 | 1600 | 20 | 100 | 200 | 20 | — | 80-85 |

*DMSO is either pure or has dissolved dye.

TABLE 2

| Name | C(1S) | O(1S) | Si(1S) | S(2P3) | N(1S) |
|---|---|---|---|---|---|
| ORMN20 | 58.5 ± 2.4 | 30.1 ± 1.2 | 9.7 ± 1.9 | 1.5 ± 0.2 | 0 |
| ORMA20 | 54.8 ± 2.4 | 30.7 ± 1.4 | 11.8 ± 1.3 | 1.3 ± 0.1 | 1.4 ± 0.3 |
| ORMA40 | 59.4 ± 0.8 | 28.4 ± 0.8 | 8.3 ± 0.4 | 1.9 ± 0.2 | 2.0 ± 0.3 |

The data provided in Table 1 indicates that the size of the nanoparticles can be 10 controlled and monodisperse nanoparticles of any size in the 10-100 nm range can be synthesized. Table 2 suggests that amino-functionality increases with increase with the amount of APTES.

### EXAMPLE 3

Determination of Entrapment Efficiency and Release Kinetics of Rh6G and HPPH from ORMOSIL Nanoparticles.

To quantify the encapsulation of amphiphilic and hydrophobic dyes, we performed a study of entrapment efficiency and release kinetics of an amphiphilic dye (Rh6G) and a hydrophobic dye (HPPH) from ORMOSIL nanoparticles. In a typical experiment, an aliquot of 500 μl of ORMN20 solution encapsulating Rh6G or HPPH was filtered through microcentrifuge filter device membranes (100-kDa cutoff) to separate the free dye from the nanoparticles. The amount of dye present in the filtrate was determined spectrophotometrically at the wavelength of absorption peak. The entrapment efficiency and release kinetics were determined by using the values for the total concentration of a dye in the system and in the filtrate, as described in ref.

We investigated the release kinetics of two types of ORMOSIL-encapsulated dyes having similar molecular weights, an amphiphilic dye (Rh6G) and a hydrophobic dye (HPPH), in an aqueous buffer system at 37° C. as shown in FIG. 2. There is a marked difference between the release behavior of the amphiphilic and the hydrophobic dyes. The amphiphilic dye shows a controlled release behavior, with an initial burst (≈10% release in the first 3 h), followed by a slow release (≈30% release in 48 h). In contrast, there is essentially no release of the hydrophobic dye (≈3% release in 48 h) for different pH values. These data suggest that by encapsulating a fluorescent hydrophobic dye, we can maintain the dye fluorescence properties of the nanoparticles over a long period. Thus, encapsulation of hydrophobic dyes in ORMOSIL nanoparticles can be used for optical tracking of nanoparticles delivery, whereas that of amphiphilic drugs/dyes can be exploited for controlled release. We also found that the entrapment efficiency of the ORMN20 nanoparticles entrapping both Rh6G and HPPH is ≈85-90%.

### EXAMPLE 4

Production and Characterization of DNA Binding to Amino functionalized ORMOSIL Nanoparticles

The amino-functionalized ORMOSIL nanoparticles form complexes with nucleic acids. The nucleic acids which can be used in the formation of the complex include one or more of the following: single and double stranded lengths of DNA, and RNA, and DNA/RNA hybrid strands.

Attachment and characterization DNA binding to the surface of the amino functionalized ORMOSIL nanoparticles was accomplished as follows. A stock solution of calf thymus (CT) DNA (1 mg/mL in 0.05 M TRIS-HCl pH 7.5) buffer was prepared and then diluted to a concentration corresponding to an optical density value of 1 at 260 nm (˜80 μM bp for CT DNA). Next, 40 μL of YOYO-1 stock solution (1 mM in DMSO) was added to 3.96 mL of this DNA solution, (final dye concentration was 10 μM). The dye-DNA solution was gently mixed, incubated in dark for 10 min and divided into two equal parts. Amino-functionalized nanoparticles (ORMA40, diameter ˜20 μm) doped with a fluorescent dye (And-10) were synthesized as described above. Freshly dialyzed ORMA40 suspension (4.0 mL) was equally divided into two cuvettes. To the first cuvette, 2 mL of buffer was added and 2 mL of DNA-YOYO-1 buffer solution added to the second cuvette. The 2 mL of DNA-YOYO-1 buffer solution was mixed with 2 mL of water in a third cuvette. The final concentration of dye and DNA in the second and third cuvettes was equivalent (5 μM of dye, 40 μM bp of CT DNA). The samples were incubated for 2 hours at 4° C. and the fluorescent emission spectra determined for each sample. Confirmation of DNA binding to the ORMOSIL nanoparticles was confirmed by fluorescence resonance energy transfer (FRET) from the excitation of And-10 to the YOYO-1 coupled to the DNA. This binding was determined to be pH dependent with a significant loss of FRET signal as the pH was decrease from 7.5 to 6.5.

Chemical and structural analyses of the ORMOSIL nanoparticles were performed by using x-ray photoelectron spectrometry and transmission electron microscopy. The chemical analysis confirmed the presence of nitrogen groups in the ORMOSIL nanoparticle preparation. The relative percentages of carbon, oxygen, nitrogen, and silicon were found to be 54.3±0.8, 29.5±0.7, 2.1±0.4, and 12.7±1.5, respectively. The presence of the organic group reduces the overall rigidity and density of the particle, which enhances the stability of such particles in aqueous systems and protects against precipitation. Optimal loading of the plasmid (pEGFP-N2) was determined to be 135 μg of DNA per ˜1014 nanoparticles. The plasmid-loaded nanoparticles retained their monodispersion and exhibit a morphology similar to that previously shown for free ORMOSIL nanoparticles (data not shown).

### EXAMPLE 5

Stability of the ORMOSIL Bound DNA

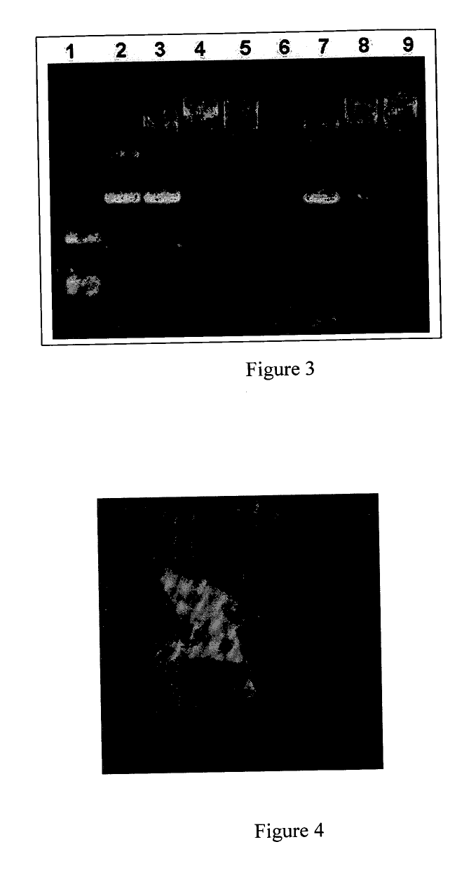

The stability of bound DNA was determined using enzymatic (DNase) digestion protocols and examination of degradation process using agarose gel electrophoresis. Briefly, 250 μL of sterile water as well as aqueous dispersion of ORMN20, ORMA20 and ORMA40 were gently mixed with four μL of plasmid pEGFP (plasmid encoding enhanced green fluorescence protein; 0.5 μg/μL) at room temperature and incubated overnight at 4° C. for the formation of DNA-nanoparticle complex. After that, 50 μLs each of the above solutions were withdrawn in duplicates in sterile eppendorf tubes. One of the sets were mixed with one μL of DNase1 (5 mg/ml) and incubated at 37° C. for 30 minutes. Next, all the solutions were run on 1% agarose at 100 volts for two hours, subsequently stained with ethidium bromide and documented using a UVP Bioimaging System. A two UV Benchtop Transilluminator, model LM-20E, was used in conjunction with an Olympus Digital Camedia C-4000 Zoom color camera having a UV filter aid lens. Documentation was completed using the Doc-It® System Software. The results are shown in FIG. 3. Upon treatment with DNase1, the free plasmid is completely digested (lane 6), whereas the plasmids bound to the amino-functionalized nanoparticles are protected (lanes 8, 9) against the same. The reason for this protection against enzymatic digestion is not yet fully understood. It has been recently suggested that this can be due to either (a) repulsion of Mg²⁺ ions (which are necessary for the enzymatic reaction) by the amino groups, or (b) a hindered access of the enzymes to the DNA which is immobilized on the nanoparticle surface, or (c) both the reasons together. Interestingly, the plasmid treated with the non-amino terminated particle (ORMN20) is also partially protected (lane 7), as it has bands corresponding to both its non-enzymatically treated counterpart (lane 3), and also some DNA fragments appearing at the bottom of the band. Therefore, these nanoparticles can also be considered as some kind of inhibitors towards the enzymatic action of DNase1 on plasmid DNA. Alternately, it is also possible that the interaction between the genetic material and the particle will not be entirely of electrostatic origin.

### EXAMPLE 6

In Vitro Transfection of Cultured Cells

The in vitro transfection of COS-1 cells was performed using the pEGFP-N. First, 20 μL of pEGFP-N2 stock solution (0.3 μg/μL in 10 mM of TRIS-HCl+1 mM of EDTA, pH 8.0) was mixed with 0.25 mL of OPTI-MEM medium and added to 0.25 mL of OPTI-MEM media containing 50 μL of ORMA40 particles (aqueous suspension). Then the contents of both microfuge tubes, the ORMA40 and plasmid mixture, was mixed and incubated at room temperature for 30 min. Finally, the resulting DNA-amino functionalized ORMOSIL complex suspension was added to a 60 mm culture plate of COS-1 cells containing 5 mL of medium and incubated for 24 hrs at 37° C. 5% CO₂. Following incubation, the transfected cells were rinsed with PBS, fresh medium added and cells immediately imaged using confocal microscopy (FIG. 4).

### EXAMPLE 7

In Vivo Transfection of Brain Cells

Plasmid expressing EGFP with the cytomegalovirus early promoter (pEGFP-N2) and mAb to EGFP were obtained from Clontech. Plasmids used to transform Escherichia coli, were isolated by using an endotoxin-free kit (Qiagen, Valencia, Calif.). Polyclonal rabbit anti-tyrosine hydroxylase (TH) Ab and BrdUrd, rabbit anti-C-terminal FGFR1 Ab, anti-BrdUrd mAb and Alexa Fluor 488-conjugated goat anti-mouse IgG, and Cy3-conjugated goat anti-rabbit IgG were purchased from commercial sources.

The pEGFP-N2 (DNA control), amino functionalized ORMOSIL (af-ORMOSIL; nanoparticle control), and amino functionalized ORMOSIL-pEGFP-N2 were injected into adult mice of both sexes by using stereotaxic surgery with equivalent concentrations of control injected materials and af-ORMOSIL-plasmid. Mice were anesthetized; an incision into the dorsal aspect of the head was made, exposing the cranium and the bregma, and a fine dental air-drill was used to drill a hole in the skull. Slow microinjection was used to deliver nanoparticles (2-6 μl containing 0.03-0.08 μg of plasmid DNA) into substantia nigra (SN) or the brain LV. Seven or 10 days after injection, mice were deeply anesthetized and perfused transcardially with PBS, followed by 4% paraformaldehyde to fix the brain tissue. The brains were removed and frozen, and 20 μm coronal or sagittal cryostat-cut sections were prepared and processed for immunocytochemistry for detection of expression of EGFP.

The fixed, free-floating brain sections were incubated in 10% NGS, followed by mouse anti-EGFP mAb (1:100 in 10% NGS) or in combination with polyclonal rabbit anti-TH Ab (1:1,000 in 10% NGS) for 72 h. After multiple rinses in PBS, sections were incubated for 2-3 h with a mixture of the appropriate secondary Abs (Alexa Fluor 488-conjugated goat anti-mouse IgG; 1:150 in 10% NGS or in combination with Cy3-conjugated goat anti-rabbit IgG; 1:600 in 10% NGS). EGFP expression was visualized by using standard fluorescence microscopy. Double immunostaining for EGFP and TH was determined from confocal images obtained in a sequential mode by using a confocal microscope (MRC 1024, Bio-Rad).

Mice transfected with ORMOSIL-pEGFP-N2 were subjected to the second stereotaxic surgery, and transfected cells were visualized in live animals by using a fibered confocal fluorescent microscopy (Cell-viZio, Discovery Technology International, Sarasota, Fla.). The CellviZio system uses a miniature fiber-optic probe (350 μm tip) that can be directly inserted into the brain tissue, permitting confocal imaging with a cellular resolution of 2.5 μm. The excitation light source is a 488-nm Argon ion laser line (Coherent, Santa Clara, Calif.), which is coupled and then focused, in sequence, through each individual microfiber. The probe was attached to a stereotaxic frame and gradually lowered into the ventricle through a 1-mm opening in the skull. The beveled tip of the probe allowed penetration in soft tissue without the need for a cannula. The resulting emitted fluorescence light, after filtering (500-650 nm), is detected by the detector housed in the main unit. The image is then reconstructed and shown on a real-time display at 12 frames per second.

The pcDNA3.1 plasmid expressing FGFR1 with the signal peptide replaced with NLS was constructed as described in ref. 28. The ORMOSIL/pFGFR1(SP-/NLS) nanoparticle complex (6 μl containing 0.08 μg of DNA) was injected into the left LV. Seven days after injection, mice were injected intraperitoneally with BrdUrd (100 mg/kg) and perfused with 4% paraformaldehyde 5 h later as described above. Consecutive sagittal brain sections, encompassing both LVs, were incubated in 4% paraformaldehyde, washed with PBS, treated with 0.5% Triton X-100, and washed again with PBS. The sections were then treated with 2M HCl at 37° C. for 15 min, neutralized in alkaline PBS (pH 8.5), washed with PBS (pH 7.4), and incubated with anti-BrdUrd mAb (1:200 in 10% NGS), followed by goat anti-mouse-Alexa Fluor 488 secondary Ab (1:150 in 10% NGS). The expression of FGFR1 in fixed sections was determined fluorescently after incubation with FGFR1 McAb6, followed by goat anti-mouse-Alexa Fluor 488 secondary Ab.

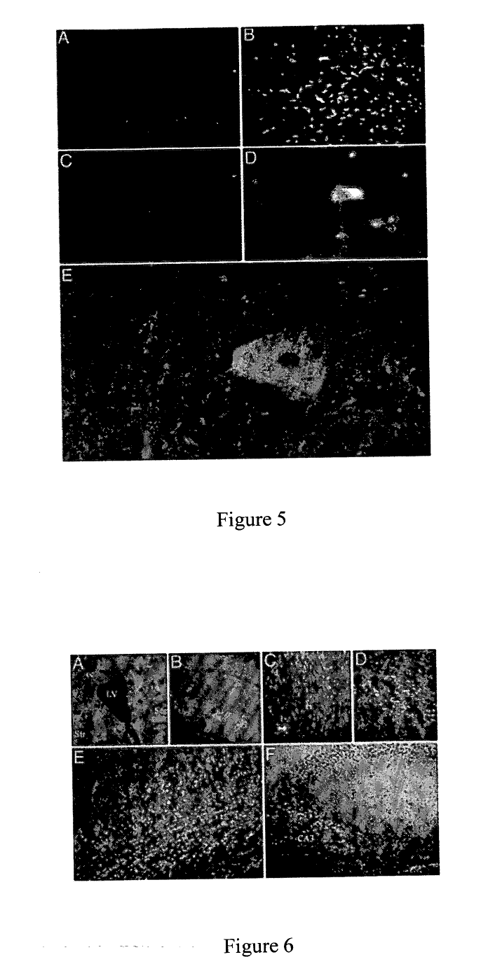

af-ORMOSIL nanoparticles, af-ORMOSIL-pEGFP-N2 nanocomplexes, and pEGFP-N2 plasmids were injected directly into the brain tissue and were examined as vehicle for gene transfer directly into the SN pars compacta (SNc), an area richly populated with neuronal cells. The presence of EGFP expression was determined by using indirect immunofluorescence with antibodies to EGFP. After the injection of the af-ORMOSIL nanoparticles, a few SNc cells exhibited a weak autofluorescence with no detectable EGFP immunoreactivity (FIG. 5A). Similar results were seen when free plasmid was injected. In contrast, injection of af-OROMSIL/pEGFP-N2 resulted in a robust EGFP expression in neuron-shaped cells (FIG. 5B), which was not observed in the absence of the primary anti-EGFP Ab (FIG. 5C). FIG. 5D shows the clear neuronal morphology of EGFP-immunopositive cells. Double immunostaining with anti-TH and anti-EGFP mAb revealed that the majority of TH-expressing cells were immunopositive for EGFP. TH immunostaining (red) was observed in peripheral cytoplasm and axonal-like processes, whereas EGFP immunoreactivity (green) was concentrated in the central cell area (FIG. 5E). The efficiency of af-ORMOSIL-mediated gene transfer was comparable with the most effective ICP4(−) herpes simplex virus 1 and was higher than that seen with herpes simplex virus 1 amplicon vector.

Gene delivery into the brain ventricular space has an advantage of minimizing damage of the brain tissue and could potentially allow expression of the transgenes in several brain structures that surround the ventricles and in cells within the ventricular wall. Seven days after the intraventricular injection of DNA-free af-ORMOSIL nanoparticles, we found no specific cellular staining within the brain by using anti-EGFP. FIG. 6A shows an area surrounding the left LV, including striatum, septum, and corpus callosum. No EGFP-immunopositive cells were present in any of the areas examined. FIG. 6B illustrates lack of staining in the left hippocampal region of DNA-free af-ORMOSIL-injected mice. In contrast, the brains of mice injected with af-ORMOSIL/pEGFP-N2 showed clear cellular EGFP immunofluorescence in the brain structures surrounding the LV. In the septum, medial to the LV, we found EGFP-expressing cells in dorsal lateral intermediate and medial septal nuclei (FIG. 6C). EGFPexpressing cells also were found within the dorsal striatum lateral to the injected ventricle (FIG. 6D). These cells displayed morphology typical of medium spiny neurons, a prevailing neuronal type in striatum. In the adjacent motor cortex, we observed EGFP-immunopositive neuron-shaped cells in several cortical layers (FIG. 6E). These EGFP-expressing cells were densely packed and displayed neuronal morphology with visible neuritic processes. In the hippocampus, EGFP-immunoreactive pyramidal neurons were present in the CA3 area (FIG. 6F).

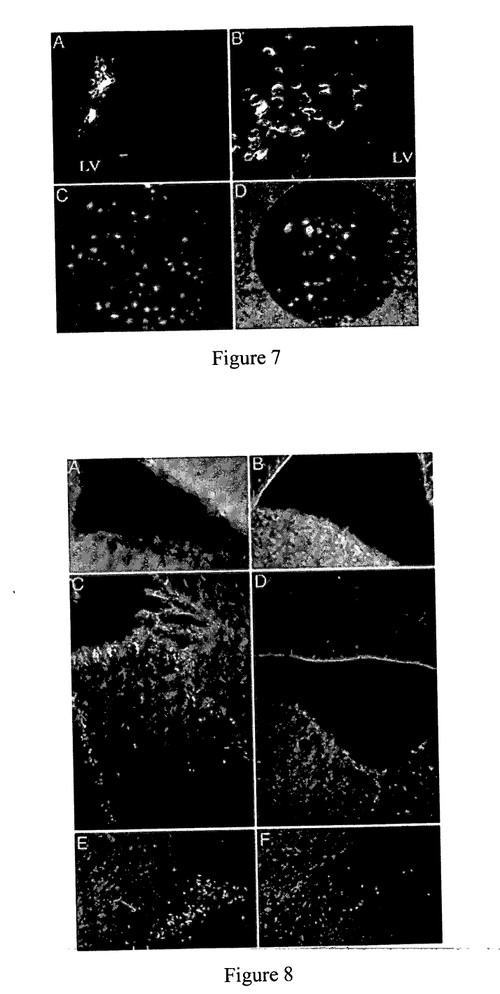

Injection of af-ORMOSIL/pEGFP-N2 complex into the LV also resulted in EGFP transfection of cells of the SVZ (FIG. 7A). FIG. 7B shows a higher magnification of transfected cells close to the ventricle. Some of these cells also have neuronal-like morphology and could represent maturating neurons. To ascertain that the immunodetected EGFP is expressed in live SVZ cells, we examined whether native EGFP fluorescence could be observed in vivo by using fiber-based confocal fluorescence microscopy (Cell-viZio). Ten days after ORMOSIL/pEGFP-N2 injection into the LV, mice were subjected to a second surgery in which the fiber-optic probe of this instrument was inserted stereotaxically into the ventricle and advanced to the inner ventricular wall. The recorded images indicated a substantial presence of transfected cells in the ventricle wall. The obtained sequences provided information about the spatial distribution of the EGFP expressing cells in animals without killing them while the probe was lowered into the ventricle. This imaging technology showed that there were more transfected fluorescent cells in the anterior/ventral region (FIG. 7C) than in the posterior region of the LV (FIG. 7D).

Given the high efficacy of ORMOSIL-mediated transfection of cells in SVZ, we examined whether this approach may be used to control the biology of these cells. To examine the role of nuclear FGFR1 in the development of brain SVZ cells in situ, we used a nonmembrane_nuclear receptor with the signal peptide replaced by NLS [FGFR1 (SP-/NLS)]. Mice received intraventricular injection of af-ORMOSIL/FGFR1(SP-/NLS) nanoparticles or DNA-free af-ORMOSIL particles, followed 10 days later by BrdUrd injection. Sagittal brain sections were immunostained with mAbs to FGFR1 or BrdUrd. FGFR1 immunostaining was found to be increased in the SVZ of mice transfected with FGFR1 (SP-/NLS) (FIG. 8 A and B). Subsequent immunostaining with anti-BrdUrd Ab revealed that a large number of cells in the SVZ (FIG. 8C) and in adjacent rostral migratory stream (FIG. 8E) incorporated BrdUrd in mice transfected with control af-ORMOSIL nanoparticles. In contrast, in mice transfected with FGFR1(SP-/NLS), only a few cells in each region were stained positive for BrdUrd (FIG. 8 D and F). This effect was not observed with FGFR1(SP-/NLS) with the tyrosine kinase domain (data not shown)

The data presented herein indicated that we have developed a synthetic system for cationic amino functionalized ORMOSIL nanoparticles which bind and protect DNA from enzymatic degradation and delivery of DNA with resulting expression of encoded protein. We have also demonstrated that in vitro and in vivo transfection studies have resulted in the efficient expression of EGFP in cells. Intraventricular injection of pEGFP-amino functionalized ORMOSIL nanoparticles resulted in selective transfection of neuronal-like cells in periventricular brain regions, striatum, septum, cortex, sub ventricular zone. Conformation of neuron transfixion was accomplished through staining for the presence of tyrosine hydroxylase on surface of transected cells. Finally, injection of pEGFP-amino functionalized ORMOSIL nanoparticles into substantia nigra region resulted in transfection of dopamine neurons as well as progenitor type cells. Based on these data, it is clear that the amino functionalized ORMOSIL nucleic acid particles can be used for delivery of the nucleic acids to desired cells. Further, these particles can also be used to elicidate the biology of stem/progenitor cells.

While this invention has been illustrated via the embodiments described herein, routine modifications will be apparent to those skilled in the art, which modifications are intended to be within the scope of the invention.

### REFERENCES

Amar, A. P., Zlokovic, B. V., Apuzzo, M. L. Neurosurgery. 52(2):402-12, discussion 412-3, 2003.
Anderson, W. F. Nature 392:25-30, 1998.
Barton, G. M., Medzhitov, R. PNAS USA 99, 14943-14945, 2002.
Blesch, A., Lu, P., Tuszynski, M. H. Brain Res Bull. 57:833-838, 2002.
Brummelkamp, T. B., Bernards, R., Agami, R. Cancer Cell. 2, 243-247, 2002.
Chang, J. W., Lee, H., Kim, E., Lee, Y., Chung, S. S., Kim, J. H. Neurosurgery. 47(4): 931-8, discussion 938-9, 2000.
Check, E. Nature 422:6927, 2003.
Das, S., Jain, T. K., Maitra, A. N. J Coll. Int. Sci. 252:82-88, 2002.
Davis, S. S. TIBTECH. 15:217-224, 1997.
Dirks, P. B., Rutka, J. T. Neurosurgery. 40(5):1000-13, discussion 1013-5, 1997.
Estin, D., Li, M., Spray, D., Wu, J. K. Neurosurgery. 44(2):361-8, discussion 368-9, 1999.
Fathallah-Shaykh, H. Archives of Neurology. 56(4):449-53, 1999.
Flotte, T. R., Laube, B. L. Chest. 120(3 Suppl):124S- 131S, 2001.
Fueyo, J., Gomez-Manzano, C., Yung, W. K., Kyritsis, A. P. Archives of Neurology. 56(4):445-8, 1999.
Glick, R. P., Lichtor, T., de Zoeten, E., Deshmukh, P., Cohen, E. P. Neurosurgery. 45(4):867-74, 1999.
Goldberg, M. S., Fleming, S. M., Palacino, J. J., Cepeda, C., Lam, H. A., Bhatnagar, A., Meloni, E. G., Wu, N., Ackerson, L. C., Klapstein, G. J., Gajendiran, M., Roth, B. L., Chesselet, M. F., Maidment, N. T., Levine, M. S., Shen, J. J Biol Chem. 278:43628-35, 2003.
Hamel, W, Westphal, M. Acta Neurochir Suppl. 88:125-35, 2003.
Helm, G. A., Alden, T. D., Sheehan, J. P., Kallmes, D. Neurosurgery. 46(5):1213-22, 2000.
Helmlinger, D., Abou-Sleymane, G., Yvert, G., Rousseau, S., Weber, C., Trottier, Y., Mandel, J. L, Devys, D. J. Neurosci. 24:1881-1887, 2004.
Henningson, C. T., Jr., Stanislaus, M. A., Gewirtz, A. M. Journal of Allergy & Clinical Immunology. 111(2 Suppl):S745-53, 2003.
Jain, T. K., Roy, I., De, T. K., Maitra, A. N. J. Am. Chem. Soc. 120:11092-11095, 1998.
Leong, K. W., Mao, H. Q., Truong-Le, V. L., Roy, K., Walsh, S. M., August, J. T. J. Contr. Rel. 53:183-193, 1998.
Lin, K. F., Chao, J., Chao, L. Hypertension. 33(1 Pt 2):219-24, 1999.
Lindsten, K., Menendez-Benito, V., Masucci, M. G., Dantuma, N. P. Nat Biotechnol. 21(8):897-902, August 2003.
Luo, J., Kaplitt, M. G., Fitzsimons, H. L., Zuzga, D. S., Liu, Y., Oshinsky, M. L., During, M. J. Science. 298(5592):425-9, 2002.
Martinez-Serrano, A., Rubio, F. J., Navarro, B., Bueno, C., Villa, A. Curr Gene Ther. 3:279-99, 2001.
Marwick, C. BMJ. 326:181, 2003.
Masliah, E., Rockenstein, E., Veinbergs, I., Sagara, Y., Mallory, M., Hashimoto, M., Mucke, L. Proc Natl Acad Sci USA. 98:12245-50, 2001.
McAllister, K., Sazani, P., Adam, M., Cho, M. J., Rubinstein, M., Samulski R. J. DeSimone J. M. J. Am. Chem. Soc. 124:15198-15207, 2002.
Morishita, R. Circulation Research. 87(9):719-21, 2000.
Nagai, N., De Mol, M., Lijnen, H. R., Carmeliet, P., Collen, D. Circulation. 99(18):2440-4, 1999.
Ooboshi, H., Ibayashi, S., Takada, J., Yao, H., Kitazono, T., Fujishima, M. Stroke. 32(4):1043-7, 2001.
Qureshi, N. H., Bankiewicz, K. S., Louis, D. N., Hochberg, F. H., Chiocca, E. A., Harsh, G. R. 4th. Neurosurgery. 46(3):663-8, discussion 668-9, 2000.
Remy, J. S., Kichler, A., Mordinov, V., Schuber, F., Behr, J. P. Proc. Natl. Acad. Sci. (USA) 92:1744-1748, 1995.
Rubin, J. B., Kieran, M. W. Current Opinion in Pediatrics. 11(1):39-46, 1999.
Rubinsztein, D. C. Trends Genet. 18:202-9, 2002.
Rutka, J. T., Taylor, M., Mainprize, T., Langlois, A., Ivanchuk, S., Mondal, S., Dirks, P Neurosurgery. 46(5):1034-51, 2000.
Sapolsky, R. M., Steinberg, G. K. Neurology. 53(9):1922-31, 1999.
Sen, L., Hong, Y. S., Luo, H., Cui, G., Laks, H. American Journal of Physiology—Heart& Circulatory Physiology. 281:H1433-41, 2001.
Shen, C., Buck, A. K., Liu, X., Winkler, M., Reske, S. N. FEBS Lett. 539:111-4, 2003.
Shi, Y. TRENDS in Genetics. 19, 9-12, 2003.
Shi, N., Pardridge, W. M. Proceedings of the National Academy of Sciences of the United States of America. 97(13): 7567-72, 2000.
Stewart, S. A., Dykxhoom, D. M., Palliser, D., Mizuno, H., Yu, E. Y., An, D. S., Sabatini, D. M., Chen, I. S., Hahn, W. C., Sharp, P. A., Weinberg, R. A., Novina, C. D. RNA. 4:493-501, 2003.
Tabbaa, S., Goulah, C., Tran, R. K., Lis, A., Korody, R., Stachowski, B., Horowitz, J. M., Torres, G., Stachowiak, E. K., Bloom, D. C., Stachowiak, M. K. Folia Morphologica (Warszawa). 59(4):221-32, 2000.
Tooyama, I., McGeer, E. G., Kawamata, T., Kimura, H., McGeer, P. L Brain Res. 656:165-168, 1994.
Weihl, C., Macdonald, R. L., Stoodley, M., Luders, J., Lin, G. Neurosurgery. 44(2):239-52, discussion 253, 1999.
Weydt, P., Hong, S. Y, Kliot, M., Moller, T. Neuroreport. 14:1051-4, 2003.
Weyerbrock, A., Oldfield, E. H. Current Opinion in Oncology. 11(3):168-73, 1999.
Yenari, M. A., Minami, M., Sun, G. H., Meier, T. J., Kunis, D. M., McLaughlin, J. R., Ho, D. Y., Sapolsky, R. M., Steinberg, G. K. Stroke. 32(4):1028-35, 2001.
Yoshimura, S., Morishita, R., Hayashi, K., Kokuzawa, J., Aoki, M., Matsumoto, K., Nakamura, T., Ogihara, T., Sakai, N., Kaneda, Y. Hypertension. 39(5):1028-34, 2002.
Zlokovic, B. V., Apuzzo, M. L. Neurosurgery. 40(4):789-803, discussion 803-4, 1997.
Zlokovic, B. V., Apuzzo, M. L. Neurosurgery. 40(4):805-12, discussion 812-3, 1997.

1. A composition comprising amino functionalized ORMOSIL nanoparticles.

2. The composition of claim 1, wherein the nanoparticles have a polynucleotide releasably complexed thereto such that the polynucleotide is rendered significantly resistant to degradation by a nuclease.

3. The composition of claim 2, wherein the polynucleotide is DNA, RNA, combinations thereof, or modifications thereof.

4. The composition of claims 3, wherein the polynucleotide is a DNA which is rendered resistant to DNAse I.

5. The composition of claim 4, wherein the DNA encodes for a gene.

6. The composition of claim 2, wherein the polynucleotide is a RNA.

7. The composition of claim 6, wherein the RNA is a siRNA.

8. The composition of claim 2, wherein the polynucleotide is a plasmid, cosmid or an artificial chromosome.

9. The composition of claim 1, wherein the ORMOSIL nanoparticles are between about 10 to 100 nm.

10. The composition of claim 9, wherein the nanoparticles are between about 20-50 nm.

11. The composition of claim 1, wherein the nanoparticles are about 30 nm.

12. A method for delivering a polynucleotide to a cell in a tissue of interest comprising contacting the tissue with a composition of claim 2.

13. The method of claim 12, wherein the polynucleotide is DNA, RNA, combinations thereof, or modifications thereof.

14. The method of claim 13, wherein the nanoparticles are between about 10 to 100 nm diameter.

15. The method of claim 14, wherein the nanoparticles are between 20-50 nm in diameter.

16. The method of claim 15, wherein the nanoparticles are about 30 nm in diameter.

17. The method of claim 12, wherein the polynucleotide is a plasmid, cosmid, or an artificial chromosome.

18. The method of claim 17, wherein the plasmid comprises a nucleotide sequence encoding a gene.

19. The method of claim 12, wherein the tissue is brain tissue.
